(19) United States
(12) Patent Application Publication
(10) Pub. No.: US 2022/0015695 A1
(43) Pub. Date: Jan. 20, 2022

(54) SLEEP REACTIVITY MONITORING BASED SLEEP DISORDER PREDICTION SYSTEM AND METHOD

(71) Applicant: KONINKLIJKE PHILIPS N.V., Eindhoven (NL)

(72) Inventors: Jenny MARGARITO, Eindhoven (NL); Jesse SALAZAR, Gibsonia, PA (US); Benjamin Irwin SHELLY, Pittsburgh, PA (US)

(21) Appl. No.: 17/380,160

(22) Filed: Jul. 20, 2021

Related U.S. Application Data
(60) Provisional application No. 63/054,197, filed on Jul. 20, 2020.
(60) Provisional application No. 63/216,261, filed on Jun. 29, 2021.

(51) Int. Cl. A61B 5/00; A61B 5/0205; A61B 5/024; A61B 5/0533; A61B 5/08; G16H 20/00; G16H 50/30

(52) U.S. Cl. CPC A61B 5/4815; A61B 5/0006; A61B 5/0022; A61B 5/4809; A61B 5/02055; A61B 2562/0219; A61B 5/02416; A61B 5/0533; A61B 5/0816; G16H 20/00; G16H 50/30; A61B 5/02405

(57) ABSTRACT

An apparatus and method for predicting the occurrence of sleep disorders and particularly insomnia, by long term monitoring of daily habits causing stress and sleep reactivity, and by coaching for correcting behaviors that can trigger the sleep disorder's occurrence and suggest interventions to mitigate the problem.

### CROSS-REFERENCE TO PRIOR APPLICATIONS

This application claims the benefit of U.S. Provisional Application No. 63/054,197, filed on 20 Jul. 2020 and 63/216,261, filed on 29 Jun. 2021. These applications are hereby incorporated by reference herein.

### BACKGROUND OF INVENTION

### 1. Field of the Invention

The present invention pertains to a system and method for reducing insomnia in a patient and, in particular, to an apparatus and method for predicting the occurrence of sleep disorders, and particularly insomnia, by long term monitoring of daily habits causing stress and sleep reactivity in conjunction with predisposing factors in insomnia, and by coaching for correcting behaviors that can trigger the sleep disorder's occurrence and suggesting interventions to mitigate the problem.

### 2. Description of the Related Art

Insomnia is among the most common sleep disorders in US. About 25 percent of Americans experience acute insomnia each year. Predisposing, precipitating, and perpetuating factors play a role in determining the occurrence and perpetuation of insomnia over time. Among the precipitating factors, stress has been shown to have a major influence in the development of insomnia, especially in subjects who are genetically predisposed. Such subjects usually manifest a disrupted sleep as response to acute daily stress, thus exhibiting what is called sleep reactivity.

In the U.S., between 50 and 70 million adults have a sleep disorder. According to literature, insomnia is the most common specific sleep disorder, with short-term issues reported by about 30% of adults, and with chronic insomnia reported by 10% of adults [“https://www.sleepassociation.org/about-sleep/sleep-statistics” (Online)]. Insomnia is defined by the presence of an individual's report of difficulty with sleep, reflected by a difficulty in falling asleep, staying asleep, or nonrestorative sleep [T. Roth, “Insomnia: definition, prevalence, etiology, and consequences,” Journal of clinical sleep medicine: JCSM: official publication of the American Academy of Sleep Medicine, 2007]. Several models have recently been developed for describing the theoretical perspectives on the etiology and pathophysiology of insomnia. One of the most known one is the “3-P model” that describes predisposing, precipitating, and perpetuating factors relevant to the development and maintenance of insomnia [D. J. e. a. Buysse, “A neurobiological model of insomnia,” Drug Discovery Today: Disease Models, pp. 129-137, 2011]. Predisposing factors include genetic, physiological, or psychological diatheses that confer differential susceptibility to individuals. Precipitating factors include physiological, environmental, or psychological stressors that push an individual over a hypothetical insomnia threshold to produce acute symptoms. Perpetuating factors include behavioral, psychological, environmental, and physiological factors that prevent the individual from re-establishing normal sleep. Among the precipitating factors, daily behaviors and stress have shown to have large impact on the development of insomnia. In particular, stress is considered to be a major trigger for insomnia, especially for subjects who are genetically predisposed to it. Such subjects show an acute sleep disturbance in response to stress exposure, with the responsive relationship being known as “sleep reactivity”. In 2014, Jarrin's team assessed 1,449 lifetime good sleepers and showed that good sleepers with high sleep reactivity were at elevated risk for insomnia symptoms and chronic insomnia disorder across the following two years than those with low sleep reactivity [D. C. e. a. Jarrin, “Temporal stability of the ford insomnia response to stress test (first),” Journal of Clinical Sleep Medicine 12.10, 2016]. The factors mainly responsible for stress are excessive workload or physical activity, caffeine intake, and impactful personal life events. However, as noted elsewhere herein, the sensitivity to such stress factors and the physiological response differ for different individuals mostly because of the predisposing factors. Biologically, stress has been shown to modify the Autonomic Nervous System (ANS) response by increasing the sympathetic activity, while decreasing the parasympathetic activity. Such variation is reflected in the Heart Rate Variability (HRV) signal, which loses power in the high frequency band, determined by the parasympathetic system, while increasing power in the low frequency band, determined by the sympathetic system. The monitoring of the ANS activity through the detection of HRV changes has therefore been commonly used to determine and quantify the stress level of a patient. It is noted that the term “patient”, as employed herein, can entail any type of a consumer or end user, without limitation.

Several solutions exist for sleep disorders detection and diagnosis of insomnia. However, no solutions are available for predicting the occurrence of a specific sleep disorder or for determining the daily habits/factors that contribute most to the development of the specific sleep disorder. A first attempt at capturing daily habits to determine a sleep condition has been covered in R. e. a. Shouldice, “METHODS AND SYSTEMS FOR SLEEP MANAGEMENT”, 2019, and U.S. Pat. No. 10,376,670. In such work, a nonspecific sleep disorder was targeted and no particular attention was given to the overnight body response. Improvements thus would be desirable.

### SUMMARY OF THE INVENTION

Accordingly, it is an object of the present invention to provide an improved system and method for reducing insomnia in a patient that overcome the shortcomings of conventional systems and methods for reducing insomnia. This object is achieved according to one embodiment of the disclosed and claimed concept by providing an apparatus and method system and method for reducing insomnia in a patient and, in particular, to an apparatus and method for predicting the occurrence of sleep disorders, and particularly insomnia, by long term monitoring of factors such as daily habits causing stress and sleep reactivity in conjunction with predisposing factors in insomnia, and by coaching for correcting behaviors that can trigger the sleep disorder's occurrence and suggesting interventions to mitigate the problem.

The determination of such factors advantageously enables the providing of recommendations for behavioral changes aimed at preventing actual occurrence of a predicted disordered sleep condition. The disclosed and claimed concept thus advantageously provides an improved system and method for predicting the occurrence of sleep disorders, and particularly insomnia, in the context of predisposing factors in the patient for insomnia, by long term monitoring of daily habits and sleep reactivity. The system and method advantageously coach for correcting behaviors that can trigger the occurrence of the sleep disorder and suggest interventions to mitigate the problem. More information regarding predisposing factors in the patient for insomnia can be found at: https://onlinelibrary.wiley.com/doi/full/10.1111/jsr.12710.

The disclosed and claimed concept advantageously focuses on assessing the risk of developing a sleep disorder-insomnia and predicting its onset given a specific patient's response to stress factors. The early prediction allows for intervening to prevent or alleviate an occurrence of the sleep disorder by determining the main risk factors for the specific person and recommending actions for reducing the impact of such factors.

The disclosed and claimed concept advantageously provides a general system for assessing risk and predicting occurrence of a generic sleep disorder as well as a specific system for insomnia based on continuous sleep reactivity measurements. A basic implementation of the system can be said to include a measurement of at least one physiological signal, a measurement of at least one sleep-influencing factor, and a set of information about daily sleep architecture.

A general system includes:
physiological signals: a number of sensor units for monitoring one or more physiological signals;
influencing factors: a number of mechanisms for monitoring type and/or intensity of factors affecting stress and/or sleep, such as a sensor for monitoring physical workload, a diary for monitoring cognitive or emotional stress (for caffeine intake, etc.);
sleep architecture: a number of mechanisms for measuring and quantifying metrics including but not limited to Sleep Onset Latency (SOL), sleep survival, spectral quantifications Cumulative Short Wave Activity (CSWA), etc., time spent in each of a number of respective sleep stages;
feature extraction block: extracts a number of statistical features from physiological/sleep data vectors;
pre-trained model: ingests the extracted features and sleep architecture data to generate a probability vector describing either the occurrence or onset of a known set of sleep disorders;
recommendation system: promotes behavioral changes or other interventions based on the calculated risk by targeting a modification of the sleep influencer that are mainly responsible for increasing the risk of a sleep disorder;
feature contribution assessment: provides a ranking of the extracted features that respectively contributed to the greatest degree to the development of a sleep disorder according to the specific model output.

As employed herein, the expression “a number of” and variations thereof shall refer broadly to any non-zero quantity, including a quantity of one. The pre-trained model is previously trained on wide population of subjects that were monitored 24/7 for long periods of time and were or were not finally diagnosed with a sleep disorder such as insomnia or others. Any learning method (Deep Net, Ensemble Trees, etc.) could be applied to generate a mathematical link between daily/night behaviors/habits and risk of developing a sleep disorder. In the feature contribution assessment, the ranking of the features is used by the recommendation engine to generate advise and recommendations for changing behaviors which, if continued, would lead to high risk of developing a specific sleep disorder. Optionally, additional information may be utilized for improving prediction accuracies, such as familiarity (e.g. a family member with a diagnosed sleep disorder), self-reported daily events and their subjective levels of associated stress, etc.

Accordingly, aspects of the disclosed and claimed concept are provided by an improved method of reducing insomnia in a patient, the general nature of which can be stated as including, during a given awake period of the patient: receiving a number of parameters of the patient that can be generally stated as including one or more of a number of awake inputs that can be generally stated as including one or more of a Heart Rate (HR), a Heart Rate Variability (HRV), a galvanic skin response, a respiration rate, a temperature, an oxygen saturation, a physical activity, a consumption of a substance, a light exposure, a workload, an emotional or physical stress, and a diary entry, and outputting from a recommendation engine a number of recommendations to the patient to reduce insomnia in the patient based at least in part upon at least a subset of the number of parameters and further based at least in part upon a degree to which each of at least some of the parameters of the at least subset has contributed to past insomnia.

Other aspects of the disclosed and claimed concept are provided by an improved system structured and configured to reduce insomnia in a patient, the general nature of which can be stated as including a processor apparatus that can be generally stated as including a processor and a storage, an input apparatus structured to provide input signals to the processor apparatus and that can be generally stated as including one or more of a number of awake inputs sensors that can be generally stated as including that can be generally stated as including one or more of a Heart Rate (HR) sensor, a Heart Rate Variability (HRV) sensor, a galvanic skin response sensor, a respiration rate sensor, a temperature, an oxygen saturation sensor, a physical activity sensor, a sensor structured to detect a consumption of a substance, a light exposure sensor, a sensor structured to detect a workload, a device structured to detect or receive an emotional or physical stress, and a diary, an output apparatus structured to receive output signals from the processor apparatus and to generate outputs, and the storage having stored therein a number of routines which, when executed on the processor, cause the system to perform a number of operations, the general nature of which can be stated as including, during a given awake period of the patient: receiving a number of parameters of the patient that can be generally stated as including one or more of a number of awake inputs that can be generally stated as including one or more of a Heart Rate (HR), a Heart Rate Variability (HRV), a galvanic skin response, a respiration rate, a temperature, an oxygen saturation, a physical activity, a consumption of a substance, a light exposure, a workload, an emotional or physical stress, and a diary entry, and outputting from a recommendation engine a number of recommendations to the patient to reduce insomnia in the patient based at least in part upon at least a subset of the number of parameters and further based at least in part upon a degree to which each of at least some of the parameters of the at least subset has contributed to past insomnia.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a depiction of an improved system in accordance with an aspect of the disclosed and claimed concept;

FIG. 2 is a depiction of a high level architecture of the system of FIG. 1;

FIG. 3 is a depiction of the relationship between stress level and sleep impairment;

FIG. 4 is a detailed depiction of the system of FIG. 1;

FIG. 5 is a further depiction of the system of FIG. 4; and

FIG. 6 is a flow chart depicting certain aspects of an improved method in accordance with the disclosed and claimed concept.

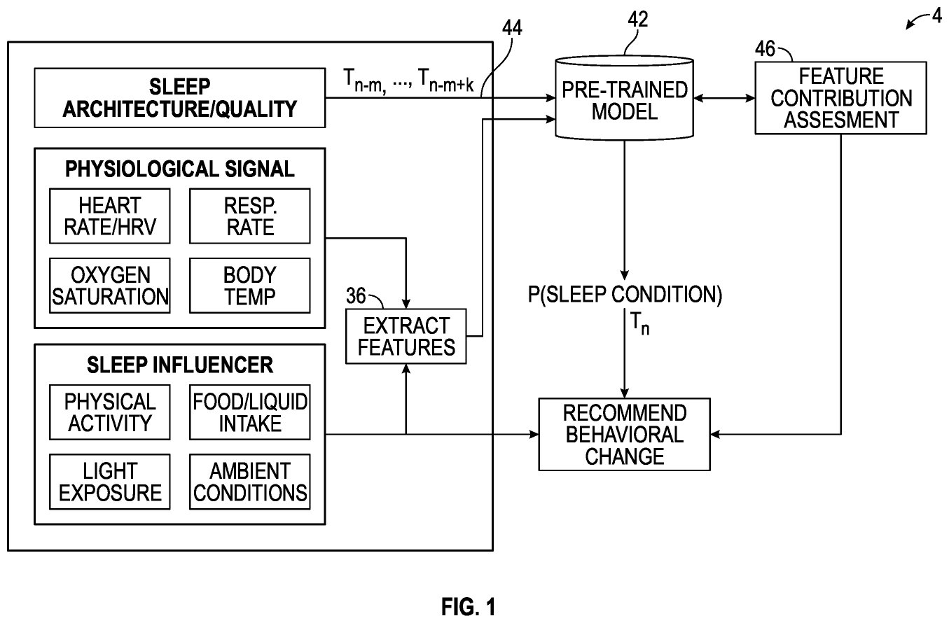

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

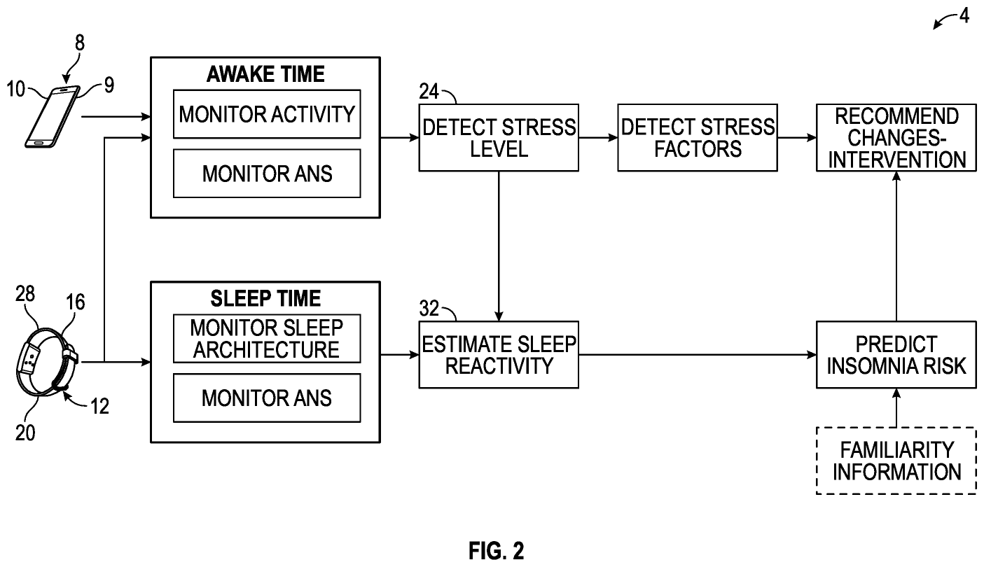

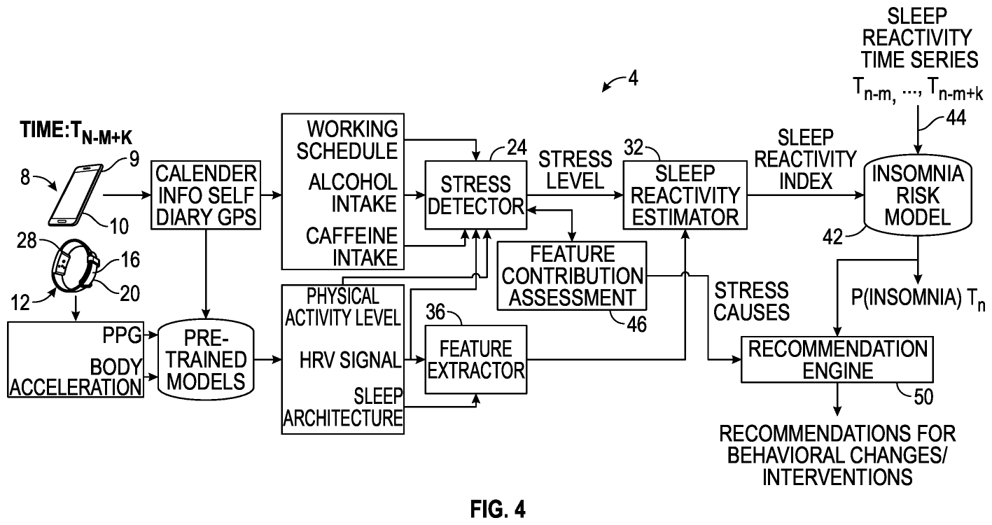

The disclosed and claimed concept advantageously provides a system 4 and a method 100 that are structured and configured for assessing the risk of a patient developing insomnia and for predicting the onset of insomnia in the patient through long term monitoring of the sleep reactivity of the patient. The sleep reactivity monitoring assesses the daily and/or event-specific stress levels in the patient, and additionally detects the subsequent physiological response to the daily and/or event-specific stress during sleep. A high-level description of the main blocks of the system 4 is shown in FIG. 2. A more detailed depiction of the system 4 is shown in FIG. 4 as including or at least interfacing with a number of elements, some or all of which can be considered to be a part of an input apparatus 6 of the system 4. These elements include a smartphone 8 that is used to collect the patient's daily activity information such as type and time of working appointments and recreational activities. The combination of a set of calendar data 9 and a set of GPS data 10 available on or accessible by smartphone 8 can be used to retrieve information about coffee break, food or alcohol intake, such as by detecting locations of pubs, restaurants, etc.

System 4 further includes or at least interfaces with a patient-worn device 12 that is equipped with a PPG 16 and a number of accelerometers 20. Patient-worn device 12 can be, for instance, a smart watch, and it is used to collect information about daily physical activity level, sleep architecture, and ANS activity from the heart rate variability signal. Alternatively, the product offered by Philips and known as the Philips Health Band device can be used as the patient-worn device 12 for sleep monitoring purposes. Pre-trained models for sleep staging, energy expenditure, and heart rate variability are known to exist and be used for building the system 4.

System 4 further includes a stress detector 24 that is used to provide a daily stress score based on information characterizing the subject's physiological status and the performed activities (physical exercise, work-related events, and/or personal events) for each specific day. Such information will be collected through the patient-worn device 12 and/or self-reporting calendar sensor 8 and GPS sensor 9. The combination of the PPG 16 and accelerometer 20 embedded in the patient-worn device 12 are used to detect the HRV signal and to therefore derive therefrom the ANS activity and the type, e.g. running, walking, sitting, etc., time (e.g. 6 PM), and intensity (e.g. averaged speed, averaged heart rate, duration of the activity, duration*speed) of the activities performed by the patient. Calendar/location or self-reported information is used to extract the working schedule to derive number, duration, and type of meetings and personal appointments. The stress detector block 24 also provides a list of factors that contribute to varying of the stress score to enable personalized recommendations for changing habits in the patient that cause the stress to increase to be provided.

The stress score can be estimated automatically from HRV data. Alternatively or in addition thereto, a Galvanic Skin Response (GSR) sensor 28 built into the patient-worn device 12 or otherwise provided can detect a GSR in the patient, and this may be used, alone or in combination with other data, to estimate stress. Alternatively or additionally, the stress level score can be based at least in part upon data provided by the patient through a questionnaire that may ask questions of the patient such as: “How would rank your stress level today in scale from 1 to 10?”). It is to be understood that the daily stress can be evaluated according to any of a variety of parameters that include any of a variety of awake inputs that may include one or more of a Heart Rate (HR), a Heart Rate Variability (HRV), a galvanic skin response, a respiration rate, a temperature, an oxygen saturation, a physical activity, a consumption of a substance, a light exposure, a workload, an emotional or physical stress, and a diary entry, for example and without limitation. Other awake inputs can be contemplated.

System 4 further includes a sleep reactivity estimator 32 that combines the stress level of the day with characteristics of the sleep architecture characterized by a number of sleep-related features that are extracted from the HRV signal via a feature extractor 36. In order to capture the ANS activity, which reflects the patient's response to stress, the HRV signal is analyzed in the frequency domain. The following features are extracted by the feature extractor 36 from the Power Spectrum Density of the HRV signal:
ULF power—Absolute power of the ultra-low-frequency band (≤0.003 Hz);
VLF power—Absolute power of the very-low-frequency band (0.0033-0.04 Hz);
LF peak—Peak frequency of the low-frequency band (0.04-0.15 Hz);
LF power—Absolute power of the low-frequency band (0.04-0.15 Hz);
LF power—Relative power of the low-frequency band (0.04-0.15 Hz) in normal units;
LF power—Relative power of the low-frequency band (0.04-0.15 Hz);
HF peak—Peak frequency of the high-frequency band (0.15-0.4 Hz);
HF power—Absolute power of the high-frequency band (0.15-0.4 Hz);
HF power—Relative power of the high-frequency band (0.15-0.4 Hz) in normal units;
HF power—Relative power of the high-frequency band (0.15-0.4 Hz); and
LF/HF %—Ratio of LF-to-HF power.

More details about HRV data processing that are usable in conjunction with the disclosed and claimed concept are set forth in, for example, F. a. J. P. G. Shaffer, “An overview of heart rate variability metrics and norms.,” Frontiers in public health 5, p. 258, 2017.

A number of features are extracted from the sleep architecture data and are usable to describe sleep characteristics. These extracted features can include:
SE: sleep efficiency (%);
REM %: percentage of sleep in Rapid Eye Movement (REM) sleep (%);
N3%: percentage of sleep in deep sleep/N3(%);
N3: number of minutes in N3 (min);
SOL: Sleep Onset Latency (min);
WASO: Wake After Sleep Onset (hours); and
TST: Total Sleep Time (min).

Likewise, this set of features can be extended to any other characteristic that are extracted from the sleep architecture data [A. e. a. Roebuck, “A review of signals used in sleep analysis,” Physiological measurement 35.1, 2013]. Alternatively or additionally, sleep characteristics can be extracted based at least in part upon patient self-reporting via a sleep diary or the like.

Sleep characteristics are used in order to quantify the degree of sleep impairment. Examples of measures of sleep impairment include SOL, WASO, (1-SE), (8 hrs—TST), and others. Other measures of sleep impairment can be defined by patient dissatisfaction with sleep (e.g. on a Likert scale) or other subjective metrics.

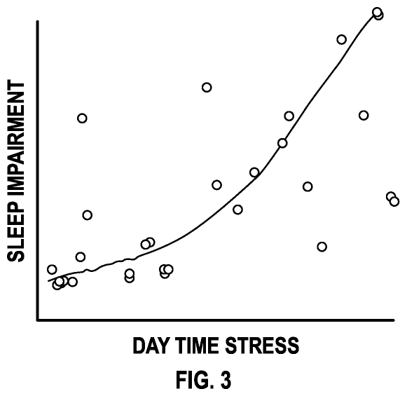

With reference to FIG. 3, daytime stress level is then compared to the degree of sleep impairment. Sleep reactivity is based at least in part upon the strength of the relationship between daytime stress and the resultant/subsequent sleep impairment. In some embodiments, the relationship is defined as a linear curve fit, and the sleep reactivity is the slope of the line. Other equations, including non-linear equations are also contemplated to define sleep reactivity. A higher slope thus defines a higher sleep reactivity measure. In FIG. 3, each data point corresponds to the stress value a given day and the sleep impairment on a subsequent night, with the line defining the best fit curve. The expression “night” is used in an exemplary fashion without limitation, and it is an example of a period of attempted sleep by the patient. As sleep reactivity can change over time and may, for instance, increase in response to chronic stress or to decrease in response to mindfulness meditation program, the disclosed and claimed concept may advantageously use data from the prior twenty-one days and nights to define the current sleep reactivity score, although other time durations such as one month, two months, six months, one week, etc., are alternatively usable.

System 4 further includes an insomnia risk model 42 that receives as an input 44 a sleep reactivity index time series generated from tₙ₋ₘ to tₙ₋₍ₘ₋ₖ₎ for estimating the risk of developing insomnia at time tₙ with following constraints:

n>=1

m,k>0

m+k<n.

System 4 further includes a feature contribution assessment module 46. For any given prediction/inference, the feature contribution assessment module 46 is a logical unit that implements an algorithm for model interpretability. One such example algorithm includes but is not limited to the SHAP method (SHapley Additive exPlanations), which takes a model-agnostic, game theoretic approach to explaining the output of a machine learning model [“https://github.com/slundberg/shap,” (Online)]. The output vector quantifies the contribution level of each input feature to a particular prediction for a given input vector. In the disclosed and claimed concept, the ranking of the various features is used by a recommendation engine 50 of the system 4 to generate advice and recommendations for changing behaviors that, if continued, would increase sleep reactivity and eventually lead to high risk of developing insomnia.

The recommendation engine 50 receives a number of inputs that may include the causes of the stress provided by the stress detector 24 and/or an ordered list of the degree to which each input feature has contributed to a particular inference/prediction. The recommendation engine 50 draws recommendations from a predefined recommendation set contained therein that includes one or more of:
a number of recommendations which attempt to directly modify an input signal/feature (e.g. for a subjective input of mg of caffeine, a recommendation to reduce caffeine consumption) and/or a prioritization of these recommendations is informed by the feature contribution assessment module 46; and/or
recommendations which attempt to modify behaviors or signals which are not directly received as inputs to the pre-trained model (e.g. modifying exercise intensity, given a system which only receives temporal sleep information and bed time heart rate), wherein each such recommendation defines the input features which it attempts to indirectly modify. The feature contribution assessment module 46 is then used to prioritize/sort these recommendations based on the contribution of each such input feature to the most recent prediction for the patient.

The recommendation engine 50 generates a number of outputs to the patient that propose behavioral changes or interventions such as any one or more of the following exemplary suggestions:
reduce amount of caffeine intake during the day;
engage in physical activities earlier in the day;
avoid late work meetings;
avoid taking naps during the day;
reduce sleep time tonight and tomorrow night;
engage in paced breathing exercise when feeling stressed; and
begin and end the day with a mindfulness exercise.

The Philips Health Band device can be used to monitor an electroencephalograph (EEG) signal that can be used in conjunction with or can take the place of the HRV signal to increase the accuracy of the sleep architecture data as well as to detect the existence of a number of sleep arousal events. Information about the incidence of sleep arousal events can be used to enhance the sleep reactivity quantification.

Sleep reactivity, insomnia risk, and behavioral recommendations can be output in any of a variety of fashions using an output apparatus 54 of the system 4. In this regard, the output apparatus 54 can interface with the smartphone 8 to enable the sleep reactivity, insomnia risk, and behavioral recommendations to be presented to the patient via a smartphone application that is executed at least in part on the smartphone 8. Sleep reactivity and insomnia risk are advantageously trended over time and presented to the patient along with recommendations in order to improve engagement and to reduce the risk of sleep impairment.

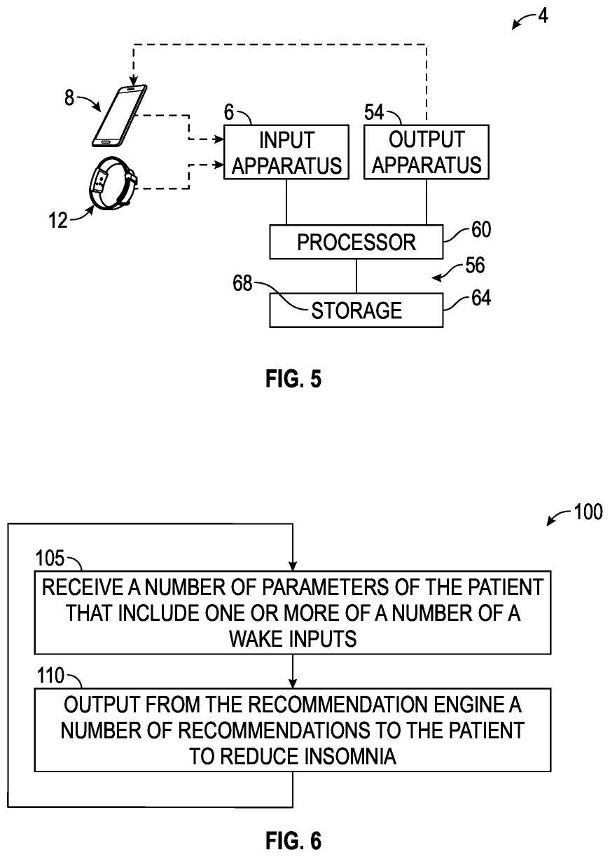

The apparatus 4 is depicted in a schematic fashion in FIG. 5. Apparatus 4 can be employed in performing the improved method 100 that is likewise in accordance with the disclosed and claimed concept and at least a portion of which is depicted in a schematic fashion in FIG. 6. Apparatus 4 can be characterized as including a processor apparatus 56 that can be said to include a processor 60 and a storage 64 that are connected with one another. Storage 64 is in the form of a non-transitory storage medium that has stored therein a number of routines 68 that are likewise in the form of a non-transitory storage medium and that include instructions which, when executed on processor 60, cause apparatus 4 to perform certain operations such as are mentioned elsewhere herein.

The input apparatus 6 of system 4 provides input signals to processor 60, and output apparatus 54 receives output signals from processor 60 and provides outputs that are detectable by the patient, such as audible outputs, visual outputs, and the like without limitation, potentially via a smartphone application on smartphone 8.

Certain aspects of the improved method 100 noted hereinbefore are depicted in the flow chart shown generally in FIG. 6. For each of a plurality of periods of attempted sleep by the patient, the method 100 performs the operations depicted generally in FIG. 6. For instance, the method 100 includes receiving, as at 105, with the system 4 a number of parameters of the patient, with the number of parameters including one or more of the number of awake inputs that can include, for example and without limitation, a Heart Rate (HR), a Heart Rate Variability (HRV), a galvanic skin response, a respiration rate, a temperature, an oxygen saturation, a physical activity, a consumption of a substance, a light exposure, a workload, an emotional or physical stress, a diary entry, and any of a variety of other awake inputs. Processing continues with the outputting, as at 110, from the recommendation engine 50 a number of recommendations to the patient to reduce the risk of a sleep impairment such as insomnia. As noted elsewhere herein, the number of recommendations are typically based at least in part upon one or more of the parameters and are further based at least in part upon a degree to which each of these parameters has contributed to past insomnia. The various recommendations can be output directly by the output apparatus 54 as audible outputs, visual outputs, and the like, or can additionally or alternatively be output via a smart phone app on the smartphone 8. Variations and other benefits will be apparent.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of reducing insomnia in a patient, comprising:
during a given awake period of the patient:
receiving a number of parameters of the patient that comprise one or more of a number of awake inputs comprising one or more of a Heart Rate (HR), a Heart Rate Variability (HRV), a galvanic skin response, a respiration rate, a temperature, an oxygen saturation, a physical activity, a consumption of a substance, a light exposure, a workload, an emotional or physical stress, and a diary entry; and
outputting from a recommendation engine a number of recommendations to the patient to reduce insomnia in the patient based at least in part upon at least a subset of the number of parameters and further based at least in part upon a degree to which each of at least some of the parameters of the at least subset has contributed to past insomnia.

2. The method of claim 1, further comprising outputting from the recommendation engine the number of recommendations further based at least in part upon a degree to which each of at least some of the parameters of the at least subset has contributed to past insomnia

3. The method of claim 2, further comprising:
outputting from the recommendation engine as the number of recommendations a plurality of recommendations, at least some of the recommendations each being related to a corresponding parameter and being ranked in order of the degree to which the corresponding parameter has contributed to past insomnia.

4. The method of claim 2, further comprising:
determining a stress level based at least in part upon at least a portion of the number of parameters;
inputting the stress level to a sleep reactivity estimator engine;
during a given sleep period of the patient subsequent to the given awake period, receiving a number of sleep architecture inputs of the patient and determining therefrom one or more of a Sleep Onset Latency (SOL), a Sleep Efficiency (SE), a Wake After Sleep Onset (WASO), a Total Sleep Time (TST), a sleep survival, a spectral quantification of sleep, and an amount of time spent in each of a number of sleep stages, determining a sleep impairment based at least in part upon the number of sleep architecture inputs, and inputting the sleep impairment to the sleep reactivity estimator engine; and
storing the stress level and the sleep impairment in the sleep reactivity estimator engine.

5. The method of claim 4, further comprising determining the stress level based upon at least one of the HRV, the GSR, and a subjective input from the patient.

6. The method of claim 4, further comprising outputting from the recommendation engine the number of recommendations additionally based at least in part upon a sleep reactivity index from the sleep reactivity estimator engine, the sleep reactivity index being based at least in part upon the stress level.

7. The method of claim 6, further comprising:
determining an insomnia probability using an insomnia risk model and based at least in part upon the sleep reactivity index; and
outputting from the recommendation engine the number of recommendations further based at least in part upon the insomnia probability.

8. The method of claim 6, further comprising determining the sleep reactivity index based at least in part upon a frequency domain analysis of the HRV and an analysis of a number of features that are extracted from the power spectrum density of the HRV.

9. The method of claim 6, further comprising determining the sleep reactivity index based at least in part upon an electroencephalogram (EEG) input from the patient.

10. The method of claim 1, further comprising receiving as the one or more of the number of awake inputs one or more of a Global Positioning System (GPS) input, an accelerometer input, a photoplethysmogram (PPG) input, and a calendar input.

11. A system structured and configured to reduce insomnia in a patient, comprising:
a processor apparatus comprising a processor and a storage;
an input apparatus structured to provide input signals to the processor apparatus and comprising one or more of a number of awake inputs sensors comprising one or more of a Heart Rate (HR) sensor, a Heart Rate Variability (HRV) sensor, a galvanic skin response sensor, a respiration rate sensor, a temperature, an oxygen saturation sensor, a physical activity sensor, a sensor structured to detect a consumption of a substance, a light exposure sensor, a sensor structured to detect a workload, a device structured to detect or receive an emotional or physical stress, and a diary;
an output apparatus structured to receive output signals from the processor apparatus and to generate outputs; and
the storage having stored therein a number of routines which, when executed on the processor, cause the system to perform a number of operations comprising:
during a given awake period of the patient:
receiving a number of parameters of the patient that comprise one or more of a number of awake inputs comprising one or more of a Heart Rate (HR), a Heart Rate Variability (HRV), a galvanic skin response, a respiration rate, a temperature, an oxygen saturation, a physical activity, a consumption of a substance, a light exposure, a workload, an emotional or physical stress, and a diary entry; and
outputting from a recommendation engine a number of recommendations to the patient to reduce insomnia in the patient based at least in part upon at least a subset of the number of parameters and further based at least in part upon a degree to which each of at least some of the parameters of the at least subset has contributed to past insomnia.

12. The system of claim 11, wherein the operations further comprise outputting from the recommendation engine the number of recommendations further based at least in part upon a degree to which each of at least some of the parameters of the at least subset has contributed to past insomnia

13. The system of claim 12, wherein the operations further comprise:
outputting from the recommendation engine as the number of recommendations a plurality of recommendations, at least some of the recommendations each being related to a corresponding parameter and being ranked in order of the degree to which the corresponding parameter has contributed to past insomnia.

14. The system of claim 12, wherein the operations further comprise:
determining a stress level based at least in part upon at least a portion of the number of parameters;
inputting the stress level to a sleep reactivity estimator engine;
during a given sleep period of the patient subsequent to the given awake period, receiving a number of sleep architecture inputs of the patient and determining therefrom one or more of a Sleep Onset Latency (SOL), a Sleep Efficiency (SE), a Wake After Sleep Onset (WASO), a Total Sleep Time (TST), a sleep survival, a spectral quantification of sleep, and an amount of time spent in each of a number of sleep stages, determining a sleep impairment based at least in part upon the number of sleep architecture inputs, and inputting the sleep impairment to the sleep reactivity estimator engine; and
storing the stress level and the sleep impairment in the sleep reactivity estimator engine.

15. The system of claim 14, wherein the operations further comprise determining the stress level based upon at least one of the HRV, the GSR, and a subjective input from the patient.

16. The system of claim 14, wherein the operations further comprise outputting from the recommendation engine the number of recommendations additionally based at least in part upon a sleep reactivity index from the sleep reactivity estimator engine, the sleep reactivity index being based at least in part upon the stress level.

17. The system of claim 16, wherein the operations further comprise:
determining an insomnia probability using an insomnia risk model and based at least in part upon the sleep reactivity index; and
outputting from the recommendation engine the number of recommendations further based at least in part upon the insomnia probability.

18. The system of claim 16, wherein the operations further comprise determining the sleep reactivity index based at least in part upon a frequency domain analysis of the HRV and an analysis of a number of features that are extracted from the power spectrum density of the HRV.

19. The system of claim 16, wherein the operations further comprise determining the sleep reactivity index based at least in part upon an electroencephalogram (EEG) input from the patient.

20. The system of claim 11, wherein the operations further comprise receiving as the one or more of the number of awake inputs one or more of a Global Positioning System (GPS) input, an accelerometer input, a photoplethysmogram (PPG) input, and a calendar input.
